(19) United States
(12) Patent Application Publication
(10) Pub. No.: US 2026/0248712 A1
(43) Pub. Date: Aug. 27, 2026

(54) COMPOSITION

(71) Applicant: SHISEIDO COMPANY, LTD., Chuo-ku, Tokyo (JP)

(72) Inventors: Ryushi FUKUHARA, Tokyo (JP); Chi Je PARK, Tokyo (JP); Cheen Aik ANG, Tokyo (JP); Akio NASU, Tokyo (JP)

(73) Assignee: SHISEIDO COMPANY, LTD., Chuo-ku, Tokyo (JP)

(21) Appl. No.: 18/879,416

(22) PCT Filed: Jul. 14, 2023
(86) PCT No.: PCT/JP2023/026054

§ 371 Date: Dec. 27, 2024

(30) Foreign Application Priority Data
Jul. 28, 2022 (JP) 2022-120662

(51) Int. Cl. A61K 8/891; A61K 8/06; A61K 8/27; A61K 8/29; A61K 8/31; A61K 8/37; A61K 8/85; A61Q 17/04

(52) U.S. Cl. CPC A61K 8/891; A61K 8/062; A61K 8/27; A61K 8/29; A61K 8/31; A61K 8/37; A61K 8/85; A61Q 17/04; A61K 2800/591; A61K 2800/651

(57) ABSTRACT

To provide a composition in which UV scattering agent particles are well dispersed and which is less likely to cause caking.

A composition, particularly a cosmetic composition in which UV scattering agent particles are dispersed in a mixture of an oily medium and polyhydroxystearic acid, which satisfies the following conditions (I) to (III):
condition (I): in the composition, the oily medium contains a polar oil and a silicone oil, or contains a polar oil, a hydrocarbon oil, and a silicone oil, the content of the silicone oil is 55 parts by mass or more based on a total amount of 100 parts by mass of the oily medium, and the content of the UV scattering agent particles is 10 parts by mass or more based on a total amount of 100 parts by mass of the oily medium,
condition (II): when calculation is performed after removal of volatile components of the oily medium, the content of the silicone oil after the removal of the volatile components is 40 parts by mass or less based on a total amount of 100 parts by mass of the oily medium after the removal of the volatile components, and
condition (III): the mass ratio between the polyhydroxystearic acid and the UV scattering agent particles (the mass of the polyhydroxystearic acid/the mass of the UV scattering agent particles) is 0.02 or more.

### TECHNICAL FIELD

The present invention relates to a composition, particularly a cosmetic composition, and more particularly, a sunscreen cosmetic composition, in which UV scattering agent particles are dispersed in a mixture of an oily medium and polyhydroxystearic acid.

### BACKGROUND ART

In the field of sunscreen cosmetics, UV scattering agent particles are mainly used as UV protection agents. Such UV scattering agent particles are dispersed in an oily medium and then added in many cases. As the oily medium, it is generally known to use, for example, polar oils such as ester oils and UV absorbing agents, hydrocarbon oils, silicone oils, or combinations thereof.

In cosmetics such as sunscreen compositions, in order to improve the dispersibility of UV scattering agent particles, generally, the composition of the cosmetics has been adjusted.

For example, PTL 1 discloses a water-in-oil type emulsion cosmetic in which, when fine metal oxide particles are dispersed in an oil phase containing a non-silicone oiling agent, fine metal oxide particles whose surfaces are treated with trialkoxyalkylsilane are used, and additionally, polyhydroxystearic acid is used as a dispersing agent, thereby improving the dispersibility of the fine metal oxide particles in the oil phase, and additionally, the aqueous component is emulsified with a specific silicone surfactant, which provides excellent dispersibility and stability over time for the fine metal oxide particles, and good usability with no stickiness and good spreadability. More specifically, the cosmetic of PTL 1 is a water-in-oil type emulsion cosmetic including the following components (A) to (E): (A) fine metal oxide particles whose surfaces are treated with trialkoxyalkylsilane, (B) polyhydroxystearic acid, (C) a non-silicone oiling agent, (D) a polyoxyalkylene/alkyl co-modified silicone having an HLB of 2 to 7 and/or a polyglycerin/alkyl co-modified silicone having an HLB of 2 to 7, (E) and an aqueous component.

In addition, PTL 2 discloses an oily dispersion in which zinc oxide or titanium oxide and ethylhexyl methoxycinnamate, which aggregated previously when mixed, do not undergo an aggregation phenomenon, and can be dispersed in a system, and an emulsion composition using this oily dispersion. More specifically, the oily dispersion of PTL 2 is an oily dispersion containing zinc oxide or titanium oxide, ethylhexyl methoxycinnamate and polyhydroxy fatty acids.

### CITATION LIST

### Patent Literature

[PTL 1] Japanese Patent Application Publication No. 2013-071931
[PTL 2] Japanese Patent Application Publication No. 2016-222602

### SUMMARY OF INVENTION

### Technical Problem

However, in compositions such as sunscreen cosmetics, even if the dispersibility of UV scattering agent particles is improved, a phenomenon in which UV scattering agent particles having precipitated due to being left to stand or the like stick together and do not return to their original dispersion state even when shaken (that is, particle caking phenomenon) may occur.

The present invention is made in order to improve the above circumstance, and an object of the present invention is to provide a composition in which UV scattering agent particles are well dispersed and which is less likely to cause caking.

### Solution to Problem

The present invention that achieves the above object is as follows.

### <Aspect 1>

A composition, particularly a cosmetic composition in which UV scattering agent particles are dispersed in a mixture of an oily medium and polyhydroxystearic acid, which satisfies the following conditions (I) to (III):
condition (I)
in the composition,
the oily medium comprises a polar oil and a silicone oil, or comprises a polar oil, a hydrocarbon oil, and a silicone oil,
the content of the silicone oil is 55 parts by mass or more based on a total amount of 100 parts by mass of the oily medium, and
the content of the UV scattering agent particles is 10 parts by mass or more based on a total amount of 100 parts by mass of the oily medium,
condition (II)
when calculation is performed after removal of volatile components of the oily medium,
the content of the silicone oil after the removal of the volatile components is 40 parts by mass or less based on a total amount of 100 parts by mass of the oily medium after the removal of the volatile components, and
condition (III)
the mass ratio between the polyhydroxystearic acid and the UV scattering agent particles (the mass of the polyhydroxystearic acid/the mass of the UV scattering agent particles) is 0.02 or more.

### <Aspect 2>

The composition according to Aspect 1,
wherein, according to the condition (I),
in the composition,
the content of the silicone oil is 55 parts by mass or more and 83 parts by mass or less based on a total amount of 100 parts by mass of the oily medium.

### <Aspect 3>

The composition according to Aspect 2,
wherein, according to the condition (I),
in the composition,
the content of the polar oil is 10 parts by mass or more based on a total amount of 100 parts by mass of the oily medium, and
the content of the hydrocarbon oil is 20 parts by mass or less based on a total amount of 100 parts by mass of the oily medium, and
wherein, according to the condition (II),
when calculation is performed after removal of volatile components of the oily medium,
the content of the polar oil after the removal of the volatile components is 20 parts by mass or more based on a total amount of 100 parts by mass of the oily medium after the removal of the volatile components,
the content of the hydrocarbon oil after the removal of the volatile components is 70 parts by mass or less based on a total amount of 100 parts by mass of the oily medium after the removal of the volatile components, and
the content of the silicone oil after the removal of the volatile components is 40 parts by mass or less based on a total amount of 100 parts by mass of the oily medium after the removal of the volatile components.

### <Aspect 4>

The composition according to any one of Aspects 1 to 3,
wherein, according to the condition (II),
when calculation is performed after removal of volatile components of the oily medium,
the content of the polyhydroxystearic acid is 1.0 part by mass or more and 30 parts by mass or less based on a total amount of 100 parts by mass of the oily medium the removal of the volatile components and the polyhydroxystearic acid.

### <Aspect 5>

The composition according to any one of Aspects 1 to 4,
wherein the UV scattering agent particles are at least one selected from the group consisting of titanium oxide, zinc oxide, iron oxide, and cerium oxide.

### <Aspect 6>

The composition according to any one of Aspects 1 to 5,
wherein the silicone oil includes a linear silicone oil.

### <Aspect 7>

The composition according to any one of Aspects 1 to 6,
wherein the polar oil does not comprise a UV absorbing agent.

### <Aspect 8>

The composition according to any one of Aspects 1 to 7, which is an oily cosmetic composition.

### <Aspect 9>

The composition according to any one of Aspects 1 to 7, which is a water-in-oil type emulsion cosmetic composition,
wherein the content of water is less than 30 mass %.

### <Aspect 10>

The composition according to any one of Aspects 1 to 7, which is an oil-in-water type emulsion cosmetic composition.

### <Aspect 11>

The composition according to any one of Aspects 1 to 10, which is a sunscreen cosmetic composition.

### Advantageous Effects of Invention

According to the present invention, it is possible to provide a composition in which UV scattering agent particles are well dispersed and which is less likely to cause caking.

In addition, in the composition of the present invention, the UV scattering agent particles are well dispersed, and after the composition is applied to the skin, white cast caused by UV scattering agent particles is inhibited, the transparency is high and a UV protection effect is good. In addition, the composition of the present invention is less likely to cause caking, despite good dispersibility of the UV scattering agent particles. Therefore, the composition of the present invention is suitable as a cosmetic composition, particularly as a sunscreen cosmetic composition.

### BRIEF DESCRIPTION OF DRAWINGS

FIG. 1 is a diagram showing change over time in absorbance in a UVB region (290 nm) for a composition of Example 14.

FIG. 2 is a diagram showing change over time in visible light transmittance at 490 nm for the composition of Example 14.

### DESCRIPTION OF EMBODIMENTS

Hereinafter, embodiments of the present invention will be described in detail. Here, the present invention is not limited to the following embodiments, and various modifications can be performed within the scope of the gist of the invention.

### <<Composition>>

The composition of the present invention is a composition, particularly a cosmetic composition in which UV scattering agent particles are dispersed in a mixture of an oily medium and polyhydroxystearic acid, which satisfies the following conditions (I) to (III):
condition (I)
in the composition,
the oily medium contains a polar oil and a silicone oil, or contains a polar oil, a hydrocarbon oil, and a silicone oil,
the content of the silicone oil is 55 parts by mass or more based on a total amount of 100 parts by mass of the oily medium, and
the content of the UV scattering agent particles is 10 parts by mass or more based on a total amount of 100 parts by mass of the oily medium,
condition (II)
when calculation is performed after removal of volatile components of the oily medium,
the content of the silicone oil after the removal of the volatile components is 40 parts by mass or less based on a total amount of 100 parts by mass of the oily medium after the removal of the volatile components, and
condition (III)
the mass ratio between the polyhydroxystearic acid and the UV scattering agent particles (the mass of the polyhydroxystearic acid/the mass of the UV scattering agent particles) is 0.02 or more.

The inventors conducted extensive studies, and found that, in a composition in which UV scattering agent particles are dispersed, it is possible to improve the dispersibility of UV scattering agent particles, but a caking phenomenon occurs once a certain dispersion state is reached.

On the other hand, when the composition of the present invention satisfies the above conditions (I) to (III), before use (that is, during storage), the dispersibility of the UV scattering agent particles is not excessively improved, and thereby the occurrence of a caking phenomenon is inhibited. Thus, after the composition is applied to the skin, the composition changes due to volatilization of a part of the oily medium contained, and thereby the dispersibility of the UV scattering agent particles is improved. As a result, after the composition of the present invention is applied to the skin, the UV scattering agent particles are well dispersed, and thereby white cast caused by aggregation of the UV scattering agent particles is inhibited, the transparency is high, a UV protection effect is good and caking is less likely to occur.

### <Oily Medium>

The composition of the present invention contains an oily medium.

The oily medium according to the present invention contains a polar oil and a silicone oil or contains a polar oil, a hydrocarbon oil, and a silicone oil. That is, the oily medium of the present invention essentially contains a polar oil and a silicone oil, and may or may not contain a hydrocarbon oil. Hereinafter, respective oily media will be described in detail.

### (Polar Oil)

The composition of the present invention contains a polar oil. Here, the “polar oil” is an oil having a high polarity among oils that can be used in cosmetics other than a silicone oil, and for example, is an oil having an IOB value of 0.10 or more, 0.11 or more, 0.12 or more, or 0.13 or more. In addition, the IOB value of the polar oil according to the present invention may be 0.50 or less, 0.45 or less, or 0.40 or less. Here, the IOB value is an abbreviation of inorganic/organic balance, which is a value indicating a ratio between the inorganic value and the organic value, and is an index indicating the degree of polarity of an organic compound. Specifically, the IOB value is expressed as an IOB value=inorganic value/organic value. Regarding each of “inorganic value” and “organic value,” “inorganic values,” and “organic values” are set according to various atoms or functional groups, for example, the “organic value” for one carbon atom in a molecule is 20, and the “inorganic value” for one hydroxyl group is 100, and the IOB value of the organic compound can be calculated by summing the “inorganic values,” and “organic values” of all atoms and functional groups in the organic compound (for example, refer to “Organic Conceptual Diagram—Basics and Applications,” Yoshio Koda, pp. 11 to 17, Sankyo Shuppan, 1984).

In the composition of the present invention, as the polar oil, a volatile polar oil may be used, a non-volatile polar oil may be used or a mixture thereof may be used. Most polar oils used in cosmetics are non-volatile.

Here, in the present invention, the “volatile” oily medium is an oily medium that exhibits a volatile content of more than 20% when left at 30° C. for 120 minutes under atmospheric pressure. On the other hand, in the present invention, the “non-volatile” oily medium is an oily medium that exhibits a volatile content of 20% or less when left at 30° C. for 120 minutes under atmospheric pressure.

Here, in the present invention, the volatility can be evaluated by the following method. 2 mg/cm² of an oil is added dropwise onto a 5 mm square acrylic resin substrate, and the weight is measured before and after the sample is left on a precision balance at 30° C. for 120 minutes. The amount of the oil lost due to volatilization is calculated from the difference in weight between before and after being left for 120 minutes, and the ratio (%) with respect to the applied weight is evaluated.

In the composition of the present invention, the content of the polar oil is not particularly limited, and in consideration of the dispersibility of the UV scattering agent particles, the content of the polar oil may be, for example, 10 parts by mass or more based on a total amount of 100 parts by mass of the oily medium. More specifically, in the composition of the present invention, the content of the polar oil may be 10 parts by mass or more, 15 parts by mass or more, 20 parts by mass or more, 25 parts by mass or more, or 30 parts by mass or more, and may be 45 parts by mass or less, 40 parts by mass or less, 35 parts by mass or less, or 30 parts by mass or less based on a total amount of 100 parts by mass of the oily medium.

In addition, when calculation is performed after removal of volatile components of the oily medium, the content of the polar oil after the removal of the volatile components may be 20 parts by mass or more, 25 parts by mass or more, 30 parts by mass or more, 35 parts by mass or more, 45 parts by mass or more, 50 parts by mass or more, 55 parts by mass or more, 60 parts by mass or more, 65 parts by mass or more, 70 parts by mass or more, 75 parts by mass or more, 80 parts by mass or more, 85 parts by mass or more, 90 parts by mass or more, 95 parts by mass or more, or 100 parts by mass (that is, the entire oily medium after vaporizing (removal) of the volatile components is a polar oil after the removal of the volatile components) based on a total amount of 100 parts by mass of the oily medium after the removal of the volatile components.

Here, in the present invention, “when calculation is performed after removal of volatile components of the oily medium” can be considered to correspond to the change in composition of the oily medium caused by volatilization of the volatile components contained in the oily medium after the composition of the present invention is applied to the skin.

In the present invention, the polar oil may contain, for example, an ester oil, a UV absorbing agent, or a mixture thereof, but in order to obtain a non-sticky feel when used, it is preferable that the polar oil do not contain a UV absorbing agent.

### Ester Oil

In the present invention, specific examples of ester oils that can be used as polar oils include tripropylene glycol dineopentanoate, isononyl isononanoate, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isoisocetyl stearate, cholesteryl 12-hydroxystearate, cetyl ethylhexanoate, ethylene glycol di-2-ethylhexanoate, dipentaerythritol fatty acid ester, N-alkyl glycol monoisostearate, neopentyl glycol dicaprylate, diisostearyl malate, glycerin di-2-heptylundecanoate, trimethylolpropane tri-2-ethylhexanoate, trimethylolpropane triisostearate, pentaerythritol tetra-2-ethylhexanoate, triethylhexanoin (glycerin tri-2-ethylhexanoate), glycerin trioctanoate, glycerin triisopalmitate, trimethylolpropane triisostearate, cetyl 2-ethylhexanoate, 2-ethylhexyl palmitate, glycerin trimyristate, tri-2-heptylundecanoic acid glyceride, castor oil fatty acid methyl ester, oleyl oleate, acetoglyceride, 2-heptylundecyl palmitate, diisobutyl adipate, N-lauroyl-L-glutamate-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, and triethyl citrate, but the present invention is not limited thereto.

In the present invention, the amount of the ester oil contained in the polar oil may be, for example, 50 parts by mass or more, 60 parts by mass or more, 70 parts by mass or more, 80 parts by mass or more, 90 parts by mass or more, or 100 parts by mass (that is, the entire polar oil is an ester oil) based on a total amount of 100 parts by mass of the polar oil.

### UV Absorbing Agent

In the present invention, specific examples of UV absorbing agents include benzoic acid derivatives, salicylic acid derivatives, cinnamic acid derivatives, dibenzoylmethane derivatives, β,β-diphenylacrylate derivatives, benzophenone derivatives, benzylidene camphor derivatives, phenylbenzimidazole derivatives, triazine derivatives, phenylbenzotriazole derivatives, anthranil derivatives, imidazoline derivatives, benzalmalonate derivatives, and 4,4-diarylbutadiene derivatives, but the present invention is not limited thereto.

It is preferable that the polar oil of the present invention do not contain a UV absorbing agent, but when a UV absorbing agent is contained, the content thereof may be, for example, 0.05 parts by mass or more, 0.1 parts by mass or more, or 0.5 parts by mass or more, and may be 50 parts by mass or less, 40 parts by mass or less, 30 parts by mass or less, 20 parts by mass or less, 10 parts by mass or less, 5.0 parts by mass or less, or 1.0 part by mass or less based on a total amount of 100 parts by mass of the polar oil.

### (Silicone Oil)

The composition of the present invention contains a silicone oil. Here, the “silicone oil” is an oil having a main framework formed by siloxane bonds among oils that can be used in cosmetics.

In the composition of the present invention, as the silicone oil, a volatile silicone oil may be used, a non-volatile silicone oil may be used, or a mixture thereof may be used. In order to improve the effect of the present invention, the composition of the present invention preferably contains a volatile silicone oil.

In the composition of the present invention, for example, in order to improve the caking resistance, the content of the silicone oil is 55 parts by mass or more based on a total amount of 100 parts by mass of the oily medium. More specifically, the content of the silicone oil may be 55 parts by mass or more, 60 parts by mass or more, 65 parts by mass or more, or 70 parts by mass or more, and may be 83 parts by mass or less, 80 parts by mass or less, 75 parts by mass or less, 70 parts by mass or less, 65 parts by mass or less, or 60 parts by mass or less based on a total amount of 100 parts by mass of the oily medium.

In addition, when calculation is performed after removal of volatile components of the oily medium, the content of the silicone oil after the removal of the volatile components may be 40 parts by mass or less, 35 parts by mass or less, 30 parts by mass or less, 25 parts by mass or less, 20 parts by mass or less, 15 parts by mass or less, 10 parts by mass or less, 5.0 parts by mass or less, 1.0 part by mass or less, or 0 parts by mass (that is, the entire silicone oil is volatile), and may be 0.1 parts by mass or more, 0.5 parts by mass or more, 1.0 part by mass or more, 5.0 parts by mass or more, or 10 parts by mass or more based on a total amount of 100 parts by mass of the oily medium after the removal of the volatile components. When calculation is performed after removal of volatile components of the oily medium, the content of the silicone oil within the above range is preferable because the dispersibility of the UV scattering agent particles is improved after the composition of the present invention is applied to the skin.

In the present invention, the silicone oil may be an acyclic silicone oil (that is, a chain silicone oil, preferably a linear silicone oil) or a cyclic silicone oil.

In the present invention, specific examples of silicone oils include, for example, chain silicone oils such as polydimethylsiloxane (dimethicone), methylphenylpolysiloxane, and methylhydrogenpolysiloxane, cyclic silicone oils such as octamethylcyclotetrasiloxane, decamethylcyclopentasiloxane, and dodecamethylcyclohexasiloxane, and mixtures of two or more of these, but the present invention is not limited thereto.

### (Hydrocarbon Oil)

The composition of the present invention may optionally contain a hydrocarbon oil. Here, the “hydrocarbon oil” is a hydrocarbon oil other than a polar oil. The composition of the present invention preferably contains a hydrocarbon oil in order to improve the compatibility between the polar oil and the silicone oil.

In the present invention, as the hydrocarbon oil, a volatile hydrocarbon oil may be used, a non-volatile hydrocarbon oil may be used, or a mixture thereof may be used. In order to improve the effect of the present invention, when the composition of the present invention contains a hydrocarbon oil, it preferably contains a volatile hydrocarbon oil.

When the composition of the present invention contains a hydrocarbon oil, the content of the hydrocarbon oil based on a total amount of 100 parts by mass of the oily medium may be 0.1 parts by mass or more, 0.5 parts by mass or more, 1.0 part by mass or more, 2.0 parts by mass or more, 3.0 parts by mass or more, 4.0 parts by mass or more, 5.0 parts by mass or more, 6.0 parts by mass or more, 7.0 parts by mass or more, or 8.0 parts by mass or more, and may be 20 parts by mass or less, 19 parts by mass or less, 18 parts by mass or less, 17 parts by mass or less, 16 parts by mass or less, 15 parts by mass or less, or 14 parts by mass or less.

In addition, if the composition of the present invention contains a hydrocarbon oil, when calculation is performed after removal of volatile components of the oily medium, the content of the hydrocarbon oil after the removal of the volatile components may be 70 parts by mass or less, 60 parts by mass or less, 50 parts by mass or less, 40 parts by mass or less, 30 parts by mass or less, 20 parts by mass or less, 10 parts by mass or less, 5.0 parts by mass or less, 1.0 part by mass or less, or 0 parts by mass (that is, the entire hydrocarbon oil is volatile), and may be 0.1 parts by mass or more, 0.5 parts by mass or more, 1.0 part by mass or more, 5.0 parts by mass or more, or 10 parts by mass or more based on a total amount of 100 parts by mass of the oily medium after the removal of the volatile components.

In the present invention, specific examples of hydrocarbon oils include decane, dodecane, isododecane, isohexadecane, undecane, tridecane, liquid paraffin, olefin oligomer, squalane, squalene, paraffin, and mixtures of two or more thereof, but the present invention is not limited thereto.

### <Polyhydroxystearic Acid>

The composition of the present invention contains polyhydroxystearic acid.

Polyhydroxystearic acid mainly has a function of uniformly dispersing UV scattering agent particles in an oily medium. Polyhydroxystearic acid is a compound oligomerized by forming ester bonds with hydroxystearic acid, commercially available products include, for example, HS oligomer 600 (commercially available from Hokoku Corporation), and Salacos HS-6C (commercially available from The Nisshin OilliO Group, Ltd.), and these can be used. In addition, the degree of polymerization of polyhydroxystearic acid is not particularly limited, and may be, for example, 4 to 8.

In the composition of the present invention, the mass ratio between the polyhydroxystearic acid and the UV scattering agent particles (the mass of the polyhydroxystearic acid/the mass of the UV scattering agent particles) is 0.02 or more. Here, in consideration of the dispersion effect, the mass ratio between the polyhydroxystearic acid and the UV scattering agent particles is preferably 0.02 or more. More specifically, in the composition of the present invention, the mass ratio between the polyhydroxystearic acid and the UV scattering agent particles may be, for example, 0.02 or more, 0.04 or more, 0.06 or more, 0.08 or more, 0.10 or more, 0.11 or more, 0.12 or more, 0.13 or more, 0.14 or more, 0.15 or more, 0.16 or more, 0.17 or more, 0.18 or more, 0.19 or more, or 0.20 or more, and may be 0.50 or less, 0.40 or less, or 0.30 or less.

In addition, when calculation is performed after removal of volatile components of the oily medium, the content of the polyhydroxystearic acid may be, for example, 1.0 part by mass or more, 1.5 parts by mass or more, 2.0 parts by mass or more, 2.5 parts by mass or more, 3.0 parts by mass or more, 3.5 parts by mass or more, 4.0 parts by mass or more, 4.5 parts by mass or more, 5.0 parts by mass or more, 5.5 parts by mass or more, 6.0 parts by mass or more, 6.5 parts by mass or more, 7.0 parts by mass or more, 7.5 parts by mass or more, 8.0 parts by mass or more, 8.5 parts by mass or more, 9.0 parts by mass or more, 9.5 parts by mass or more, or 10 parts by mass or more, and may be 30 parts by mass or less, 25 parts by mass or less, 20 parts by mass or less, 15 parts by mass or less, 14 parts by mass or less, 13 parts by mass or less, 12 parts by mass or less, 11 parts by mass or less, or 10 parts by mass or less based on a total amount of 100 parts by mass of the oily medium after the removal of the volatile components and the polyhydroxystearic acid.

### <UV Scattering Agent Particles>

In the composition of the present invention, the UV scattering agent particles are dispersed in a mixture of an oily medium and polyhydroxystearic acid. More specifically, it is thought that, when a state in which polyhydroxystearic acid is adsorbed and oriented at the interface between the UV scattering agent and the oily medium is formed, the inter-particle interaction between UV scattering agent particles in the oily medium is controlled, and thereby the dispersibility and caking resistance of the UV scattering agent particles are improved.

The content of the UV scattering agent particles is 10 parts by mass or more based on a total amount of 100 parts by mass of the oily medium. In order to obtain a higher SPF, the content of the UV scattering agent particles may be 10 parts by mass or more, 20 parts by mass or more, or 25 parts by mass or more based on a total amount of 100 parts by mass of the oily medium. Here, the upper limit of the amount of the UV scattering agent particles dispersed in the oily medium is not particularly limited, and for example, the content of the UV scattering agent particles may be 200 parts by mass or less, 100 parts by mass or less, 50 parts by mass or less, or 30 parts by mass or less based on a total amount of 100 parts by mass of the oily medium.

In the present invention, the UV scattering agent particles are not particularly limited as long as they have a UV protection effect, and may be, for example, at least one selected from the group consisting of titanium oxide, zinc oxide, iron oxide, and cerium oxide.

The UV scattering agent particles may or may not be hydrophobized. Here, the hydrophobic treatment may be performed using a known surface treatment agent for hydrophobization, and examples thereof include a fluorine compound treatment, a silicone treatment, a silicone resin treatment, a pendant treatment, a silane coupling agent treatment, a titanium coupling agent treatment, an oiling agent treatment, an N-acylated lysine treatment, a polyacrylic acid treatment, a metal soap treatment, a dextrin fatty acid ester treatment, an amino acid treatment, an inorganic compound treatment, a plasma treatment, a mechanochemical treatment, a silane compound treatment, and a silazane compound treatment. Among these treatments, in consideration of dispersion stability and the like, a treatment with silicone or a silicone resin, a treatment with a silane compound or a silazane compound, and a metal soap treatment with stearic acid/aluminum hydroxide are preferable.

The average primary particle size of the UV scattering agent particles is not particularly limited, and may be, for example, 5 nm or more, 10 nm or more, or 15 nm or more, and may be 200 nm or less, 100 nm or less, or 50 nm or less. Here, the “average primary particle size” may be determined as a diameter of a circle equivalent to the projected area of primary particles in a TEM image.

The shape of the UV scattering agent particle is not particularly limited, and examples thereof include a spherical shape, a plate shape, a rod shape, a spindle shape, a needle shape, and an irregular shape.

### <<Method of Producing Composition>>

The method of producing the composition of the present invention is not particularly limited, and by a known method, the composition of the present invention can be produced by mixing the above components and dispersing UV scattering agent particles. Examples of dispersion devices include media type dispersion devices such as a bead mill, a sand mill, a ball mill, and a paint shaker, high-speed stirring machines such as a homogenizer and a disperser, and media-less dispersion devices such as a high-pressure dispersing machine and an ultrasonic dispersing machine. There are horizontal and vertical type bead mills, and either type can be used. Generally, a dispersion composition having good storage stability can be obtained by lowering the temperature of the dispersion during dispersion. For example, dispersion may be performed so that the temperature of the dispersion is 10° C. to 40° C.

### <<Cosmetic>>

The composition of the present invention can be suitably used as a cosmetic or a raw material thereof. That is, the present invention particularly provides a cosmetic composition and more particularly a sunscreen cosmetic composition.

The form of the cosmetic composition of the present invention is not particularly limited. Therefore, the cosmetic composition of the present invention may be an oil-in-water type emulsion cosmetic composition, a water-in-oil type emulsion cosmetic composition, an oily cosmetic composition based on an oiling agent or the like.

For example, when the composition of the present invention is a water-in-oil type emulsion cosmetic composition, it may further contain water. In this case, the content of water is not particularly limited, and may be, for example, 1.0 mass % or more, 5.0 mass % or more, or 10 mass % or more, and may be less than 30 mass %, 25 mass % or less, 20 mass % or less, or 10 mass % or less with respect to the entire composition.

In addition, in addition to the above components, the cosmetic composition of the present invention may further contain an optional aqueous component other than water that can be used in the field of cosmetics, or other components.

Examples of aqueous components other than water include monohydric alcohols such as ethanol, polyhydric alcohols such as butylene glycol, dipropylene glycol, propanediol, pentylene glycol, hexanediol, and glycerin, polyalkylene glycols such as polyethylene glycol and polypropylene glycol or copolymers thereof, inorganic salts such as sodium chloride and magnesium chloride or mixtures thereof, but the present invention is not limited thereto.

The cosmetic composition of the present invention may further contains, for example, surfactants, moisturizing agents, higher alcohols, sequestering agents, natural and synthetic polymers, water-soluble and oil-soluble polymers, various extract liquids, and coloring agents such as organic dyes, and other components such as preservatives, antioxidants, colorants, thickeners, pH adjusting agents, fragrance agents, cooling agents, antiperspirant agents, disinfectants, skin activators, other medicinals, and various powders, but the present invention is not limited thereto.

Examples of surfactants include a lipophilic nonionic surfactant and a hydrophilic nonionic surfactant.

Here, the lipophilic nonionic surfactant is not particularly limited, and examples thereof include sorbitan fatty acid esters such as sorbitan monooleate, sorbitan monoisostearate, sorbitan monolaurate, sorbitan monopalmitate, sorbitan monostearate, sorbitan sesquioleate, sorbitan sesquiisostearate, sorbitan trioleate, diglycerol sorbitan penta-2-ethylhexylate, and diglycerol sorbitan tetra-2-ethylhexylate, glycerin polyglycerin fatty acids such as monocotton seed oil fatty acid glycerin, monoerucate glycerin, sesquioleate glycerin, monostearate glycerin, α,α′-oleic acid pyroglutamic acid glycerin, and glycerin monostearate malate, propylene glycol fatty acid esters such as propylene glycol monostearate, hydrogenated castor oil derivatives, and glycerin alkyl ethers.

The hydrophilic nonionic surfactant is not particularly limited, and examples thereof include POE sorbitan fatty acid esters such as POE sorbitan monooleate, POE sorbitan monostearate, and POE sorbitan tetraoleate, POE sorbitol fatty acid esters such as POE sorbitol monolaurate, POE sorbitol monooleate, POE sorbitol pentaoleate, and POE sorbitol monostearate, POE glycerin fatty acid esters such as POE glycerin monostearate, POE glycerin monoisostearate, and POE glycerin triisostearate, POE fatty acid esters such as POE monooleate, POE distearate, POE monodioleate, and ethylene glycol distearate, POE alkyl ethers such as POE lauryl ether, POE oleyl ether, POE stearyl ether, POE behenyl ether, POE2-octyldodecyl ether, and POE cholestanol ether, POE alkyl phenyl ethers such as POE octylphenyl ether, POE nonylphenyl ether, and POE dinonylphenyl ether, pluronic types such as Pluronic, POE/POP alkyl ethers such as POE/POP cetyl ether, POE/POP 2-decyl tetradecyl ether, POE/POP monobutyl ether, POE/POP hydrogenated lanolin, and POE/POP glycerin ether, tetra POE/tetra POP ethylenediamine condensates such as tetronic, POE castor oil hydrogenated castor oil derivatives such as POE castor oil, POE hydrogenated castor oil, POE hydrogenated castor oil monoisostearate, POE hydrogenated castor oil triisostearate, POE hydrogenated castor oil monopyroglutamic acid monoisostearate diester, and POE hydrogenated castor oil maleic acid, POE beeswax/lanolin derivatives such as POE sorbitol beeswax, alkanolamides such as coconut oil fatty acid diethanolamide, lauric acid monoethanolamide, and fatty acid isopropanolamide, POE propylene glycol fatty acid ester, POE alkylamine, POE fatty acid amide, sucrose fatty acid ester, POE nonylphenyl formaldehyde condensate, alkyl ethoxy dimethyl amine oxide, and trioleyl phosphate.

As other surfactants, for example, anionic surfactants such as fatty acid soap, higher alkyl sulfate ester salts, POE triethanolamine lauryl sulfate, and alkyl ether sulfate, cationic surfactants such as alkyl trimethyl ammonium salts, alkyl pyridinium salts, alkyl quaternary ammonium salts, alkyl dimethyl benzyl ammonium salts, alkylamine salts, and polyamine fatty acid derivatives, and amphoteric surfactants such as imidazoline-based amphoteric surfactants and betaine-based surfactants may be added within the range that does not cause stability and skin irritation problems.

The moisturizing agent is not particularly limited, and examples thereof include xylitol, sorbitol, maltitol, chondroitin sulfate, hyaluronic acid, mucoitin sulfate, charonin acid, atelocollagen, cholesteryl-12-hydroxystearate, sodium lactate, bile salts, dl-pyrrolidone carboxylate, short-chain soluble collagen, diglycerin (EO) PO adducts, chestnut rose extracts, yarrow extracts, and melilot extracts.

The higher alcohol is not particularly limited, and examples thereof include linear alcohols such as lauryl alcohol, cetyl alcohol, stearyl alcohol, behenyl alcohol, myristyl alcohol, oleyl alcohol, and cetostearyl alcohol, and branched alcohols such as monostearyl glycerin ether (batyl alcohol), 2-decyltetradecynol, lanolin alcohol, cholesterol, phytosterol, hexyldodecanol, isostearyl alcohol, and octyldodecanol.

The sequestering agent is not particularly limited, and examples thereof include 1-hydroxyethane-1,1-diphosphonic acid, 1-hydroxyethane-1,1-diphosphonic acid tetrasodium salts, sodium citrate, sodium polyphosphate, sodium metaphosphate, gluconic acid, phosphoric acid, citric acid, ascorbic acid, succinic acid, and edetic acid.

The natural water-soluble polymer is not particularly limited, and examples thereof include plant-based polymers such as gum arabic, tragacanth gum, galactan, guar gum, carob gum, karaya gum, carrageenan, pectin, agar, quince seed (marmello), algae colloid (brown alage extract), starch (rice, corn, potato, wheat), and glycyrrhizic acid, microorganism-based polymers such as xanthan gum, dextran, succinoglucan, and pullulan, and animal-based polymers such as collagen, casein, albumin, and gelatin.

The semi-synthetic water-soluble polymer is not particularly limited, and examples thereof include starch-based polymers such as carboxymethyl starch and methylhydroxypropyl starch, cellulose-based polymers such as methylcellulose, nitrocellulose, ethylcellulose, methylhydroxypropylcellulose, hydroxyethylcellulose, sodium cellulose sulfate, hydroxypropyl cellulose, sodium carboxymethyl cellulose (CMC), crystalline cellulose, and cellulose powder, and alginic acid-based polymers such as sodium alginate and propylene glycol alginate.

The synthetic water-soluble polymer is not particularly limited, and examples thereof include vinyl-based polymers such as polyvinyl alcohol, polyvinyl methyl ether, and polyvinylpyrrolidone, polyoxyethylene-based polymers such as polyethylene glycol 20,000, 40,000, and 60,000, polyoxyethylene/polyoxypropylene copolymer coplymerized polymers, acrylic-based polymers such as sodium polyacrylate, polyethyl acrylate, and polyacrylamide, polyglycerin, polyethylene imine, cationic polymers, carboxyvinyl polymers, alkyl-modified carboxyvinyl polymers, (hydroxyethyl acrylate/sodium acryloyldimethyl taurate) copolymers, (sodium acrylate/sodium acryloyldimethyl taurate) copolymers, (ammonium acryloyldimethyl taurate/vinyl pyrrolidone) copolymers, and (ammonium acryloyldimethyltaurate methacrylate beheneth-25) crosspolymers.

The inorganic water-soluble polymer is not particularly limited, and examples thereof include bentonite, AlMg silicate (Veegum), laponite, hectorite, and silicic anhydride.

Other medicinal components are not particularly limited, and examples thereof include vitamins such as vitamin A oil, retinol, retinol palmitate, inositol, pyridoxine hydrochloride, benzyl nicotinate, nicotinamide, DL-α-tocopherol nicotinate, magnesium ascorbate phosphate, 2-O-α-D-glucopyranosyl-L-ascorbic acid, vitamin D2 (ergocalciferol), dl-α-tocopherol, dl-α-tocopherol acetate, pantothenic acid, and biotin; hormones such as estradiol and ethynylestradiol; amino acids such as arginine, aspartic acid, cystine, cysteine, methionine, serine, leucine, and tryptophan; anti-inflammatory agents such as allantoin and azulene, and brightening agents such as arbutin; astringents such as tannic acid; and refreshing agents such as L-menthol and camphor, sulfur, lysozyme chloride, and pyridoxine chloride.

Various extract liquids are not particularly limited, and examples thereof include Houttuynia cordata extracts, Phellodendron amurense extracts, melilot extracts, lamium extracts, licorice extracts, peony extracts, soapwort extracts, loofah extracts, cinchona extracts, saxifrage extracts, Sophora flavescens extracts, nuphar extracts, fennel extracts, primrose extracts, rose extracts, rehmannia root extracts, lemon extracts, lithospermum root extracts, aloe extracts, calamus root extracts, eucalyptus extracts, horsetail extracts, sage extracts, thyme extracts, tea extracts, seaweed extracts, cucumber extracts, clove extracts, raspberry extracts, melissa extracts, carrot extracts, horse chestnut extracts, peach extracts, peach leaf extracts, mulberry extracts, knapweed extracts, hamamelis extracts, placenta extracts, thymus extracts, silk extract liquids, and licorice extracts.

Examples of various powders include luminous color pigments such as red iron oxide, yellow iron oxide, black iron oxide, mica titanium, iron oxide-coated mica titanium, and titanium oxide-coated glass flakes, inorganic powders such as mica, talc, kaolin, sericite, zinc oxide, and silica, and organic powders such as polyethylene powder, nylon powder, cross-linked polystyrene, cellulose powder, and silicone powder.

### EXAMPLES

The present invention will be described below in more detail with reference to examples, but the present invention is not limited thereto.

### Examples 1 and 2 and Comparative Examples 1 to 6

Based on the formulations shown in the following Table 1, cosmetic compositions (oily cosmetic compositions) of examples and comparative examples were prepared. Here, in Examples 1 and 2 and Comparative Examples 1 to 6, respective oily media contained a polar oil and a silicone oil, and in Table 1, the description order of the examples and comparative examples was determined based on the order of the content of the polar oil (before removing volatile components) in the oily medium.

In addition, the obtained compositions were evaluated for “ultraviolet (UV) protection efficiency” and “caking resistance” according to the following criteria. The results are shown in Table 1.

### <UV Protection Efficiency>

A certain amount (2 mg/cm²) of each composition obtained in the examples and comparative examples was added dropwise to a 50 mm square acrylic resin pseudo-substrate simulating the texture of human skin, and uniformly applied onto the substrate with a certain load (100±30 g) at a certain speed (100 mm/sec×50 reciprocations). After drying for 60 minutes, a spectrophotometer (SPECTROPHOTOMETER U-3500, commercially available from Hitachi, Ltd.) was used to transmit UV light and visible light of 280 to 500 nm through the coated substrate, and the absorbance was calculated from the transmittance measurement.

### (Evaluation Criteria)

The maximum value of absorbance in a UV light wavelength region of 280 nm to 380 nm was calculated, and the UV protection efficiency was determined according to the following evaluation criteria:
“A”: the maximum value of absorbance was 1.6 or more,
“B”: the maximum value of absorbance was 1.4 or more and less than 1.6,
“C”: the maximum value of absorbance was 1.0 or more and less than 1.4,
“D”: the maximum value of absorbance was less than 1.0.

Here, in the case of “A,” the UV protection efficiency was evaluated as being best. Here, this result suggested that the UV scattering agent particles were most efficiently dispersed in the coating film. In addition, the UV protection efficiency was evaluated as getting worse in the order from “B” to “D,” and particularly in the case of “D,” the UV protection efficiency was evaluated as being worst. Here, this result suggested that the dispersibility of the UV scattering agent particles in the coating film was getting worse.

### (Caking Resistance)

The compositions obtained in the examples and comparative examples were evaluated for caking resistance according to a precipitation evaluation test.

More specifically, 40 g of each prepared sample was filled into a 50 ml glass screw tube and left at room temperature for 24 hours. After 24 hours, each sample container was repeatedly inverted, and the state of the powder precipitated at the bottom of the container was evaluated according to the following evaluation criteria:
“A”: when no residue was observed at the bottom of the container and no powder precipitation occurred after inversion once,
“B”: when the powder residue was observed at the bottom of the container after inversion once, and the residue disappeared after stirring by inversion 1 to 10 times,
“C”: when the powder residue was observed at the bottom of the container after inversion once, and the residue disappeared after stirring by inversion 11 to 30 times,
“D”: when the powder residue was observed at the bottom of the container after inversion once, and the residue did not disappear even after stirring by inversion up to 50 times.

Here, in the case of “A,” no caking occurred, that is, the caking resistance was evaluated as being best. In addition, the caking resistance was evaluated as getting worse in the order from “B” to “D,” and particularly in the case of “D,” the caking resistance was evaluated as being worst.

TABLE 1

| | | | | Com- | Com- | Com- | Com- | | | Com- | Com- |
|---|---|---|---|---|---|---|---|---|---|---|---|
| | | | | parative | parative | parative | parative | Ex- | Ex- | parative | parative |
| | | | | Example | Example | Example | Example | ample | ample | Example | Example |
| | | | | 1 | 2 | 3 | 4 | 1 | 2 | 5 | 6 |
| Composition | UV | Stearic acid/aluminum | | 10.0 | 10.0 | 10.0 | 10.0 | 10.0 | 10.0 | 10.0 | 10.0 |
| formulation | scattering | hydroxide/titanium oxide | | | | | | | | | |
| (each | agent | Dextrin palmitate/zinc | | 10.0 | 10.0 | 10.0 | 10.0 | 10.0 | 10.0 | 10.0 | 10.0 |
| addition | particles | oxide | | | | | | | | | |
| amount is | Polyhydroxystearic acid | | | 2.5 | 2.5 | 2.5 | 2.5 | 2.5 | 2.5 | 2.5 | 2.5 |
| expressed | Oily | Polar oil | Diisopropyl | 77.5 | 62.0 | 50.4 | 38.75 | 23.25 | 19.4 | 11.6 | 0.0 |
| as mass %) | medium | | sebacate | | | | | | | | |
| | | | (non-volatile) | | | | | | | | |
| | | Silicone | Dimethicone | 0.0 | 15.5 | 27.1 | 38.75 | 54.25 | 58.1 | 65.9 | 77.5 |
| | | oil | (1.5 CS) | | | | | | | | |
| | | | (volatile) | | | | | | | | |

| | | | | | | | | | |
|---|---|---|---|---|---|---|---|---|---|
| | Sum (mass %) | 100.0 | 100.0 | 100.0 | 100.0 | 100.0 | 100.0 | 100.0 | 100.0 |
| Based on a total | Parts by mass of polar oil | 100.0 | 80.0 | 65.0 | 50.0 | 30.0 | 25.0 | 15.0 | 0.0 |
| amount of 100 | Parts by mass of silicone | 0.0 | 20.0 | 35.0 | 50.0 | 70.0 | 75.0 | 85.0 | 100.0 |
| parts by mass of | Parts by mass of hydrocarbon oil | 0.0 | 0.0 | 0.0 | 0.0 | 0.0 | 0.0 | 0.0 | 0.0 |
| the oily medium | | | | | | | | | |
| Based on a total | Parts by mass of polar oil after | 100.0 | 100.0 | 100.0 | 100.0 | 100.0 | 100.0 | 100.0 | 100.0 |
| amount of 100 | removal of volatile components | | | | | | | | |
| parts by mass of | Parts by mass of silicone after | 0.0 | 0.0 | 0.0 | 0.0 | 0.0 | 0.0 | 0.0 | 0.0 |
| the oily medium | removal of volatile components | | | | | | | | |
| after removal of | Parts by mass of hydrocarbon oil | 0.0 | 0.0 | 0.0 | 0.0 | 0.0 | 0.0 | 0.0 | 0.0 |
| volatile | after removal of volatile | | | | | | | | |
| components | components | | | | | | | | |
| Evaluation | UV protection efficiency | B | A | A | A | A | A | D | D |
| result | Caking resistance | D | D | D | D | A | A | A | A |

As is clear from the results in Table 1, it can be understood that both the cosmetic compositions of Examples 1 and 2 had excellent UV protection efficiency and caking resistance, that is, were compositions in which UV scattering agent particles were well dispersed in the coating film and

On the other hand, it can be understood that the cosmetic composition of Comparative Example 1 had a slightly low UV protection efficiency and poor caking resistance. Thus, it can be understood that none of the cosmetic compositions of Comparative Examples 2 to 6 could achieve both the UV protection efficiency and caking resistance.

### Examples 3 to 5 and Comparative Examples 7 to 11

Based on the formulations shown in the following Table 2, cosmetic compositions (oily cosmetic compositions) of the examples and comparative examples were prepared. In addition, the obtained compositions were evaluated in the same manner as in the above “ultraviolet (UV) protection efficiency” and “caking resistance” evaluations. The results are shown in Table 2.

TABLE 2

| | | | | Com- | Com- | Com- | Com- | Com- | | | |
|---|---|---|---|---|---|---|---|---|---|---|---|
| | | | | parative | parative | parative | parative | parative | Ex- | Ex- | Ex- |
| | | | | Example | Example | Example | Example | Example | ample | ample | ample |
| | | | | 7 | 8 | 9 | 10 | 11 | 3 | 4 | 5 |
| Composition | UV | | Stearic acid/aluminum | 10.0 | 10.0 | 10.0 | 10.0 | 10.0 | 10.0 | 10.0 | 10.0 |
| formulation | scattering | | hydroxide/titanium | | | | | | | | |
| (each | agent | | oxide | | | | | | | | |
| addition | particle | | Dextrin palmitate/ | 10.0 | 10.0 | 10.0 | 10.0 | 10.0 | 10.0 | 10.0 | 10.0 |
| amount is | | | zinc oxide | | | | | | | | |
| expressed | Polyhydroxystearic acid | | | 2.5 | 2.5 | 2.5 | 2.5 | 2.5 | 2.5 | 2.5 | 2.5 |
| as mass %) | Oily | Polar oil | Diisopropyl sebacate | 24.0 | 24.0 | 24.0 | 24.0 | 24.0 | 24.0 | 24.0 | 24.0 |
| | medium | | (non-volatile) | | | | | | | | |
| | | Silicone | Dimethicone | 0.0 | 10.7 | 18.7 | 26.75 | 34.8 | 42.8 | 47.1 | 53.5 |
| | | oil | (1.5 CS) (volatile) | | | | | | | | |
| | | Hydro- | Isododecane | 53.5 | 42.8 | 34.8 | 26.75 | 18.7 | 10.7 | 6.4 | 0.0 |
| | | carbon oil | (volatile) | | | | | | | | |

| | | | | | | | | | |
|---|---|---|---|---|---|---|---|---|---|
| | Sum (mass %) | 100.0 | 100.0 | 100.0 | 100.0 | 100.0 | 100.0 | 100.0 | 100.0 |
| Based on a to | Parts by mass of polar oil | 31.0 | 31.0 | 31.0 | 31.0 | 31.0 | 31.0 | 31.0 | 31.0 |
| tal amount of | Parts by mass of silicone | 0.0 | 13.8 | 24.1 | 34.5 | 44.9 | 55.2 | 60.7 | 69.0 |
| 100 parts by | Parts by mass of hydrocarbon oil | 69.0 | 55.2 | 44.9 | 34.5 | 24.1 | 13.8 | 8.3 | 0.0 |
| mass of the | | | | | | | | | |
| oily medium | | | | | | | | | |
| Based on a | Parts by mass of polar oil after removal of | 100.0 | 100.0 | 100.0 | 100.0 | 100.0 | 100.0 | 100.0 | 100.0 |
| total amount | volatile components | | | | | | | | |
| of 100 parts | Parts by mass of silicone after removal | 0.0 | 0.0 | 0.0 | 0.0 | 0.0 | 0.0 | 0.0 | 0.0 |
| by mass of the | of volatile components d | | | | | | | | |
| oily medium | Parts by mass of hydrocarbon oil after | 0.0 | 0.0 | 0.0 | 0.0 | 0.0 | 0.0 | 0.0 | 0.0 |
| after removal | removal of volatile components | | | | | | | | |
| of volatile | | | | | | | | | |
| components | | | | | | | | | |
| Evaluation | UV protection efficiency | A | A | A | A | A | A | A | A |
| result | Caking resistance | D | D | D | D | D | A | A | A |

As is clear from the results in Table 2, it can be understood that all the cosmetic compositions of Examples 3 to 5 had excellent UV protection efficiency and caking resistance, that is, were compositions in which UV scattering agent particle were well dispersed in the coating film and which were less likely to cause caking.

On the other hand, it can be understood that none of the cosmetic compositions of Comparative Examples 7 to 11 could achieve both the UV protection efficiency and caking resistance.

### Examples 6 to 13 and Comparative Example 12

Based on the formulations shown in the following Table 3, cosmetic compositions (oily cosmetic compositions) of Examples 6 to 13 and Comparative Example 12 were prepared. In addition, the obtained compositions were evaluated in the same manner as in the above “ultraviolet (UV) protection efficiency” and “caking resistance” evaluations. The results are shown in Table 3.

TABLE 3

| | | | | | | | | | | | | Com- |
|---|---|---|---|---|---|---|---|---|---|---|---|---|
| | | | | Ex- | Ex- | Ex- | Ex- | Ex- | Ex- | Ex- | Ex- | parative |
| | | | | ample | ample | ample | ample | ample | ample | ample | ample | Example |
| | | | | 6 | 7 | 8 | 9 | 10 | 11 | 12 | 13 | 12 |
| Composition | UV | | Stearic acid/aluminum | 0.0 | 0.0 | 10.0 | 10.0 | 10.0 | 10.0 | 10.0 | 10.0 | 10.0 |
| formulation | scattering | | hydroxide/titanium oxide | | | | | | | | | |
| (each | agent | | Hydrogen dimethicone/ | 10.0 | 0.0 | 0.0 | 0.0 | 0.0 | 0.0 | 0.0 | 0.0 | 0.0 |
| addition | particles | | aluminum hydroxide/ | | | | | | | | | |
| amount is | | | titanium oxide | | | | | | | | | |
| expressed | | | Untreated titanium oxide | 0.0 | 10.0 | 0.0 | 0.0 | 0.0 | 0.0 | 0.0 | 0.0 | 0.0 |
| as mass %) | | | Dextrin palmitate/zinc oxide | 10.0 | 10.0 | 0.0 | 0.0 | 0.0 | 10.0 | 10.0 | 10.0 | 10.0 |
| | | | Triethoxycaprylylsilane/ | 0.0 | 0.0 | 10.0 | 0.0 | 0.0 | 0.0 | 0.0 | 0.0 | 0.0 |
| | | | zinc oxide | | | | | | | | | |
| | | | Dimethicone/hydrogen | 0.0 | 0.0 | 0.0 | 10.0 | 0.0 | 0.0 | 0.0 | 0.0 | 0.0 |
| | | | dimethicone/zinc oxide | | | | | | | | | |
| | | | Untreated zinc oxide | 0.0 | 0.0 | 0.0 | 0.0 | 10.0 | 0.0 | 0.0 | 0.0 | 0.0 |
| | Polyhydroxystearic acid | | | 2.5 | 2.5 | 2.5 | 2.5 | 2.5 | 2.5 | 2.5 | 2.5 | 2.5 |
| | Oily | Polar | Diisopropyl sebacate | 27.5 | 27.5 | 27.5 | 27.5 | 27.5 | 0.0 | 27.5 | 24.0 | 27.5 |
| | medium | oil | (non-volatile) | | | | | | | | | |
| | | | Cetyl ethylhexanoate | 0.0 | 0.0 | 0.0 | 0.0 | 0.0 | 27.5 | 0.0 | 0.0 | 0.0 |
| | | | (non-volatile) | | | | | | | | | |
| | | Silicone | Dimethicone (1.5 CS) | 50.0 | 50.0 | 50.0 | 50.0 | 50.0 | 50.0 | 0.0 | 42.8 | 0.0 |
| | | oil | (volatile) | | | | | | | | | |
| | | | Dimethicone (1 CS) | 0.0 | 0.0 | 0.0 | 0.0 | 0.0 | 0.0 | 50.0 | 0.0 | 0.0 |
| | | | (volatile) | | | | | | | | | |
| | | | Dimethicone (6 CS) | 0.0 | 0.0 | 0.0 | 0.0 | 0.0 | 0.0 | 0.0 | 0.0 | 50.0 |
| | | | (non-volatile) | | | | | | | | | |
| | | Hydro- | Isododecane | 0.0 | 0.0 | 0.0 | 0.0 | 0.0 | 0.0 | 0.0 | 0.0 | 0.0 |
| | | carbon | (volatile) | | | | | | | | | |
| | | oil | Undecane/tridecane | 0.0 | 0.0 | 0.0 | 0.0 | 0.0 | 0.0 | 0.0 | 10.7 | 0.0 |
| | | | (volatile) | | | | | | | | | |

| | | | | | | | | | | |
|---|---|---|---|---|---|---|---|---|---|---|
| | Sum (mass %) | 100.0 | 100.0 | 100.0 | 100.0 | 100.0 | 100.0 | 100.0 | 100.0 | 100.0 |
| Based on a | Parts by mass of polar oil | 35.5 | 35.5 | 35.5 | 35.5 | 35.5 | 35.5 | 35.5 | 31.0 | 35.5 |
| total amount | Parts by mass of silicone | 64.5 | 64.5 | 64.5 | 64.5 | 64.5 | 64.5 | 64.5 | 55.2 | 64.5 |
| of 100 parts | Parts by mass of hydrocarbon oil | 0.0 | 0.0 | 0.0 | 0.0 | 0.0 | 0.0 | 0.0 | 13.8 | 0.0 |
| by mass of | | | | | | | | | | |
| the oily | | | | | | | | | | |
| medium | | | | | | | | | | |
| Based on a | Parts by mass of polar oil after removal | 100.0 | 100.0 | 100.0 | 100.0 | 100.0 | 100.0 | 100.0 | 100.0 | 35.5 |
| total amount | of volatile components | | | | | | | | | |
| of 100 parts | Parts by mass of silicone after removal | 0.0 | 0.0 | 0.0 | 0.0 | 0.0 | 0.0 | 0.0 | 0.0 | 64.5 |
| by mass of | of volatile components | | | | | | | | | |
| the oily | Parts by mass of hydrocarbon oil after | 0.0 | 0.0 | 0.0 | 0.0 | 0.0 | 0.0 | 0.0 | 0.0 | 0.0 |
| medium | removal of volatile components | | | | | | | | | |
| after removal | | | | | | | | | | |
| of volatile | | | | | | | | | | |
| components | | | | | | | | | | |
| Evaluation | UV protection efficiency | A | A | A | A | A | A | A | A | C |
| result | Caking resistance | A | A | A | A | A | A | A | A | A |

As described in Examples 6 to 10 in Table 3, it can be understood that excellent UV protection efficiency and caking resistance were obtained even when UV scattering agent particles with or without a surface treatment of titanium oxide and zinc oxide and with different types of surface treatment agents were added.

In addition, in Examples 11, 12, and 13, those in which, as the polar oil, cetyl ethylhexanoate was used in place of diisopropyl sebacate, as the silicone oil, dimethicone (1CS) was used in place of dimethicone (1.5CS), and as the hydrocarbon oil, a mixture of undecane and tridecane was used in place of isododecane were produced. It can be clearly understood that, in these Examples 11 to 13, excellent UV protection efficiency and caking resistance were obtained.

On the other hand, as described in Comparative Example 12, it can be understood that, when non-volatile dimethicone (6CS) was used as the silicone oil, the UV protection efficiency was poor.

### Example 14

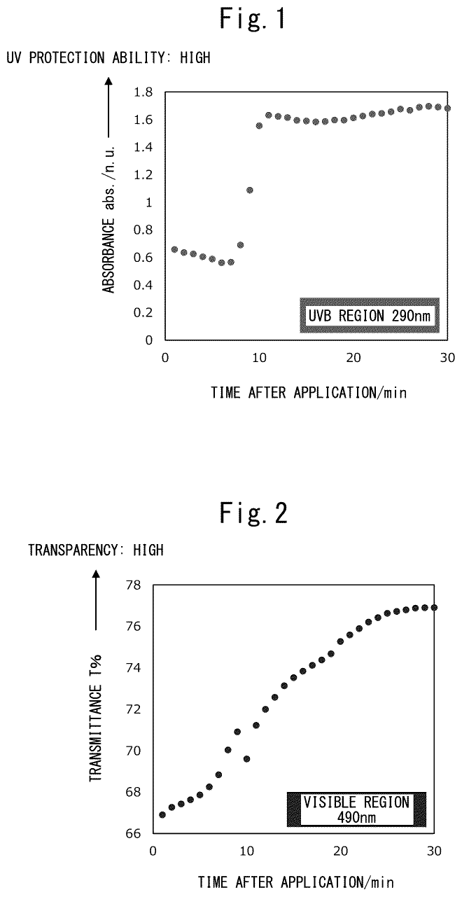

Based on the following Table 4, a cosmetic composition (oily cosmetic composition) of Example 14 was prepared. When the composition of Example 14 was observed under a stereoscopic microscope, it was confirmed that the metal oxide particles were slightly aggregated. The obtained composition was applied to an acrylic resin substrate simulating the texture of skin under a certain dropwise addition amount, load, and coating speed, and the changes over time in absorbance in the UVB region (290 nm) and the visible light transmittance at 490 nm were examined using a spectrophotometer for 30 minutes from immediately after application. The results are shown in FIG. 1 and FIG. 2.

Here, in the measurement of the UVB absorbance and visible light transmittance, the coating film was produced using the same substrate, dropwise addition amount, load, and coating speed as those used in the evaluation of “UV protection efficiency,” and measurement was performed using the same spectrophotometer.

TABLE 4

| | | | | Example |
|---|---|---|---|---|
| | | | | 14 |
| Composition | UV scattering | Stearic acid/aluminum | | 10.0 |
| formulation | agent | hydroxide/titanium oxide | | |
| (each addition | particles | Dextrin palmitate/zinc oxide | | 10.0 |
| amount is | Polyhydroxystearic acid | | | 2.5 |
| expressed as | Oily medium | Polar oil | Diisopropyl sebacate | 27.5 |
| mass %) | | | (non-volatile) | |
| | | Silicone | Dimethicone (1.5CS) | 50.0 |
| | | oil | (volatile) | |

| | | |
|---|---|---|
| | Sum (mass %) | 100.0 |
| Based on a total | Parts by mass of polar oil | 35.5 |
| amount of 100 parts | Parts by mass of silicone | 64.5 |
| by mass of the oily | Parts by mass of hydrocarbon oil | 0.0 |
| medium | | |
| Based on a total | Parts by mass of polar oil after removal of | 100.0 |
| amount of 100 parts | volatile components | |
| by mass of the oily | Parts by mass of silicone after removal of | 0.0 |
| medium after | volatile components | |
| removal of volatile | Parts by mass of hydrocarbon oil after removal | 0.0 |
| components | of volatile components | |

As is clear from the results in FIG. 1, it can be understood that the composition of Example 14 exhibited a low absorbance in the UVB region (290 nm) immediately after application, but the absorbance (that is, UV protection ability) was significantly improved after about 8 minutes. This is thought to be because, after the composition of Example 14 was applied to the skin, when a part of the oily medium contained was volatilized, the composition changed, and accordingly, the dispersibility of the UV scattering agent particles was improved, and as a result, the UV protection ability was improved.

In addition, as is clear from the results in FIG. 2, it can be understood that the visible light transmittance of the composition of Example 14 immediately after application was low, but the transmittance was significantly improved after about 8 minutes that is, the transparency was significantly improved. This is thought to be because, after the composition of Example 14 was applied to the skin, the composition changed because a part of the oily medium contained was volatilized, and accordingly, the dispersibility of the UV scattering agent particles was improved, and as a result, white cast caused by aggregation of the UV scattering agent particles was alleviated, and thus the composition had high transparency.

### Examples 15 and 16

Based on the formulations shown in the following Table 5, cosmetic compositions (water-in-oil type emulsion cosmetic compositions) of Examples 15 and 16 were prepared. In addition, the obtained compositions were evaluated in the same manner as in the above “ultraviolet (UV) protection efficiency” and “caking resistance” evaluations. The results are shown in Table 5.

TABLE 5

| | | | | Example | Example |
|---|---|---|---|---|---|
| | | | | 15 | 16 |
| Composition | UV scattering | Stearic acid/aluminum | | 10.0 | 10.0 |
| formulation | agent | hydroxide/titanium oxide | | | |
| (each addition | particle | Dextrin palmitate/zinc oxide | | 10.0 | 10.0 |
| amount is | Polyhydroxystearic acid | | | 3.0 | 3.0 |
| expressed as | Oily medium | Polar oil | Diisopropyl sebacate | 20.0 | 15.0 |
| mass %) | | | (non-volatile) | | |
| | | Silicone | Dimethicone (1.5CS) | 35.7 | 25.7 |
| | | oil | (volatile) | | |

| | | | |
|---|---|---|---|
| W/O | Sorbitan sesquiisostearate | 3.0 | 3.0 |
| emulsifying | Disteardimonium hectorite | 0.3 | 0.3 |
| agent | | | |
| Usability | Silica | 1.5 | 1.5 |
| adjustment | | | |
| powder | | | |
| Aqueous | Water | 15.0 | 30.0 |
| medium | Dipropylene glycol | 1.0 | 1.0 |
| | Phenoxyethanol | 0.5 | 0.5 |

| | | | |
|---|---|---|---|
| | Sum (mass %) | 100.0 | 100.0 |
| Based on a total | Parts by mass of polar oil | 35.9 | 36.9 |
| amount of 100 parts | Parts by mass of silicone | 64.1 | 63.1 |
| by mass of the oily | Parts by mass of hydrocarbon oil | 0.0 | 0.0 |
| medium | | | |
| Based on a total | Parts by mass of polar oil after removal of | 100.0 | 100.0 |
| amount of 100 parts | volatile components | | |
| by mass of the oily | Parts by mass of silicone after removal of | 0.0 | 0.0 |
| medium after | volatile components | | |
| removal of volatile | Parts by mass of hydrocarbon oil after removal | 0.0 | 0.0 |
| components | of volatile components | | |
| Evaluation | UV protection efficiency | A | B |
| result | Caking resistance | A | A |

As is clear from the results in Table 5, it can be understood that the water-in-oil type cosmetic composition of Example 15 had excellent UV protection efficiency and caking resistance. On the other hand, it can be understood that, in Example 16 in which the content of water was 30 mass %, the UV protection efficiency was slightly poor.

1. A composition, particularly a cosmetic composition, in which UV scattering agent particles are dispersed in a mixture of an oily medium and polyhydroxystearic acid, which satisfies the following conditions (I) to (III):
condition (I)
in the composition,
the oily medium comprises a polar oil and a silicone oil, or comprises a polar oil, a hydrocarbon oil, and a silicone oil,
the content of the silicone oil is 55 parts by mass or more based on a total amount of 100 parts by mass of the oily medium, and
the content of the UV scattering agent particles is 10 parts by mass or more based on a total amount of 100 parts by mass of the oily medium,
condition (II)
when calculation is performed after removal of volatile components of the oily medium,
the content of the silicone oil after the removal of the volatile components is 40 parts by mass or less based on a total amount of 100 parts by mass of the oily medium after the removal of the volatile components, and
condition (III)
the mass ratio between the polyhydroxystearic acid and the UV scattering agent particles (the mass of the polyhydroxystearic acid/the mass of the UV scattering agent particles) is 0.02 or more.

2. The composition according to claim 1,
wherein, according to the condition (I),
in the composition,
the content of the silicone oil is 55 parts by mass or more and 83 parts by mass or less based on a total amount of 100 parts by mass of the oily medium.

3. The composition according to claim 2,
wherein, according to the condition (I),
in the composition,
the content of the polar oil is 10 parts by mass or more based on a total amount of 100 parts by mass of the oily medium, and
the content of the hydrocarbon oil is 20 parts by mass or less based on a total amount of 100 parts by mass of the oily medium, and
wherein, according to the condition (II),
when calculation is performed after removal of volatile components of the oily medium,
the content of the polar oil after the removal of the volatile components is 20 parts by mass or more based on a total amount of 100 parts by mass of the oily medium after the removal of the volatile components,
the content of the hydrocarbon oil after the removal of the volatile components is 70 parts by mass or less based on a total amount of 100 parts by mass of the oily medium after the removal of the volatile components, and
the content of the silicone oil after the removal of the volatile components is 40 parts by mass or less based on a total amount of 100 parts by mass of the oily medium after the removal of the volatile components.

4. The composition according to claim 1,
wherein, according to the condition (II),
when calculation is performed after removal of volatile components of the oily medium,
the content of the polyhydroxystearic acid is 1.0 part by mass or more and 30 parts by mass or less based on a total amount of 100 parts by mass of the oily medium after the removal of the volatile components and the polyhydroxystearic acid.

5. The composition according to claim 1,
wherein the UV scattering agent particles are at least one selected from the group consisting of titanium oxide, zinc oxide, iron oxide, and cerium oxide.

6. The composition according to claim 1,
wherein the silicone oil includes a linear silicone oil.

7. The composition according to claim 1,
wherein the polar oil does not comprise a UV absorbing agent.

8. The composition according to claim 1, which is an oily cosmetic composition.

9. The composition according to claim 1, which is a water-in-oil type emulsion cosmetic composition,
wherein the content of water is less than 30 mass %.

10. The composition according to claim 1, which is an oil-in-water type emulsion cosmetic composition.

11. The composition according to claim 1, which is a sunscreen cosmetic composition.
